(19) United States
(12) Patent Application Publication
(10) Pub. No.: US 2023/0371791 A1
(43) Pub. Date: Nov. 23, 2023

(54) ENDOSCOPE HANDLE WITH ADJUSTABLE KNOB OFFSET

(71) Applicant: AdaptivEndo LLC, Louisville, KY (US)

(72) Inventors: Hershel E. Fancher, Georgetown, IN (US); Michael W. Goldenbogen, Xenia, OH (US); Trevor E. Powers, Louisville, KY (US); Galen R. Powers, Louisville, KY (US); Mark A. Griffin, Louisville, KY (US)

(73) Assignee: AdaptivEndo LLC, Lousiville, KY (US)

(21) Appl. No.: 18/319,963

(22) Filed: May 18, 2023

Related U.S. Application Data
(60) Provisional application No. 63/343,160, filed on May 18, 2022.

(51) Int. Cl. A61B 1/00

(52) U.S. Cl. CPC A61B 1/00105; A61B 1/00128; A61B 1/00066; A61B 1/00042

(57) ABSTRACT

An endoscope system including a first reusable hand-piece and a second reusable hand-piece. The first reusable hand-piece is a different size than the second reusable hand-piece. The first reusable hand-piece and the second reusable hand-piece are selectively attachable and detachable to the same or a different single-use shaft assembly. Varying sizes of reusable hand-piece assemblies provide for improved user ergonomics. Ergonomic variations may include different size reusable hand-piece assemblies, different size articulation control knobs, and/or different distances between the centerline of the housing of the reusable hand-piece and the knobs and/or between the backstrap of the reusable hand-piece and the knobs, customizable to user preference.

### CROSS-REFERENCE TO RELATED APPLICATION

This application claims the benefit of U.S. Provisional Application No. 63/343,160 filed May 18, 2022, which is hereby incorporated by reference. WO 2018/098465, WO 2018/191063, WO 2021/026231, WO 2022/109488 and WO 2022/125550 are hereby incorporated by reference.

### BACKGROUND OF THE DISCLOSURE

The present disclosure relates to endoscopes. Endoscopes are used in a wide variety of medical procedures to visualize internal cavities within the human body during diagnostic and/or therapeutic procedures. These procedures often occur at facilities where there are numerous clinicians practicing such procedures. If clinicians prefer different endoscopes for their procedures, the facility may stock a variety of endo scopes based simply on clinician preference, or physicians may use endoscopes which they do not prefer. Thus, there is a need for improvement in this field.

### SUMMARY OF THE DISCLOSURE

The present disclosure pertains generally to endoscopes. It is understood that many of the aspects disclosed are applicable in other technical areas, but especially in other medical devices.

Examples of endoscopic instruments are disclosed in WO 2018/098465, WO 2018/191063, WO 2021/026231, WO 2022/109488 and WO 2022/125550, all of which are hereby incorporated by reference.

The present disclosure provides an endoscope assembly comprising a reusable hand-piece and a single-use shaft assembly. The reusable hand-piece may be attachable to and detachable from the single-use shaft assembly.

Preferably, the reusable hand-piece is selectively attachable and detachable from the single-use shaft assembly such that the reusable hand-piece may be used serially with a number of single-use shaft assemblies while the single-use shaft assembly can be discarded and/or reconditioned after a single use.

Disclosed is a system with a first reusable hand-piece and a second reusable hand-piece. The first reusable hand-piece may be a different size and/or has at least one different feature than the second reusable hand-piece. Both the first reusable hand-piece and the second reusable hand-piece may be selectively attachable and detachable to a single-use shaft assembly. The first reusable hand-piece and the second reusable hand-piece are alternatively attachable to the same single-use shaft assembly. The difference in size may be a difference in cross-sectional dimension, circumference, width, length, and/or volume of the reusable hand-piece assemblies.

The first reusable hand-piece may include a housing and an articulation assembly, wherein the articulation assembly may include a first articulation control and/or a second articulation control. The first and/or second articulation control may include a knob(s).

A distance between a backstrap of the first reusable hand-piece and an axis of rotation of the first articulation control of the first reusable hand-piece may be different from a distance between a backstrap and an axis of rotation of the first articulation control of the second reusable hand-piece. Additionally or alternatively, a distance between a backstrap of the first reusable hand-piece and an axis of rotation of the second articulation control of the first reusable hand-piece may be different from a distance between a backstrap and an axis of rotation of the second articulation control of the second reusable hand-piece.

The distance between a centerline of the first reusable hand-piece and a centerline of the first articulation control (e.g., the knob) of the first reusable hand-piece may be different from the distance between a centerline of the second reusable hand-piece and first articulation control (e.g., the knob) of the second reusable hand-piece.

The first articulation control knob and/or the second articulation control knob may be selectively attachable and/or detachable to first reusable hand-piece. Advantageously, this can allow for the knobs to be interchangeable to customize the endoscope. For example, the system may include a first articulation control knob and a second first articulation control knob. The first articulation control knob may be a different size and/or may have a different number of outwardly-extending lobes than the substitute first articulation control knob. Both the first articulation control knob and a substitute first articulation control knob are alternatively and selectively attachable and detachable to the first reusable hand-piece.

Likewise, the system may include a second articulation control knob and a substitute second articulation control knob. Both the second articulation control knob and the substitute second articulation control knob are alternatively and selectively attachable and detachable to the first reusable hand-piece.

The system may further include a first brake control and/or a second first brake control. The first brake control may comprise a knob or lever, and the second brake control may comprise a knob or lever. The first brake control may be a different size than the second first brake control. Both the first brake control and a second first brake control may be selectively attachable and detachable to the first reusable hand-piece. The difference in size of the first and second brake controls may be a difference in radius and/or circumference (e.g., for knobs) and/or a difference in length.

One or more of the first articulation control, the substitute first articulation control, the second articulation control, and the substitute second articulation control may be sterilely sealed in a kit. The first articulation control and/or the second articulation control are preferably packaged separately from the substitute first control knob and/or the substitute second control knob. The first reusable hand-piece, second reusable hand-piece, and single-use shaft assembly are preferably sterilely sealed within medical packaging and may be sterilely sealed separately or in combination with one or more other of the first reusable hand-piece, second reusable hand-piece, and single-use shaft assembly.

The system may include a first articulation control and a substitute first articulation control. The first articulation control may be a knob and may have more or fewer lobes than the substitute first articulation control (e.g., knob). Both the first articulation control and a substitute first articulation control may be alternatively and selectively attachable and detachable to the first reusable hand-piece. Advantageously, this allows the user to customize the reusable hand-piece. In some embodiments, the first articulation control includes at least five lobes.

The system may include a second articulation control and a substitute second articulation control. The substitute second articulation control may be a knob and may have more or fewer lobes than the second articulation control (e.g., knob). Both the second articulation control knob and a substitute second articulation control knob may be alternatively and selectively attachable and detachable to the first reusable hand-piece. Advantageously, this allows the user to customize the reusable hand-piece. In some embodiments, the first articulation control knob includes at least five lobes.

Further disclosed is a system (e.g., a product line). The system may include a first reusable hand-piece and a second reusable hand-piece. The first reusable hand-piece may a different size than the second reusable hand-piece. Both the first reusable hand-piece and the second reusable hand-piece may alternatively and selectively attachable and detachable to a single-use shaft assembly. The reusable hand-piece may be packaged with the single-use shaft assembly in a sterilely sealed medical package. In this configuration, the handles of different sizes may be available.

In some embodiments, the difference in size is a difference in width of the reusable hand-piece assemblies. In other embodiments, the difference in size is a difference in height of the reusable hand-piece assemblies. In yet other embodiments, the difference in size is a difference in circumference of the reusable hand-piece assemblies. In other embodiments, the difference in size is a difference in internal volumes of the reusable hand-piece assemblies.

Systems may include a reusable endoscope hand-piece and a single-use endoscope shaft assembly; the reusable endoscope hand-piece selectively attachable to and detachable from the single-use endoscope shaft assembly; the reusable endoscope hand-piece including a housing and an articulation and brake assembly, wherein the articulation and brake assembly includes a first articulation control, a second articulation control, and an articulation brake for selectively locking rotation of said second articulation control relative to the housing of said reusable endoscope hand-piece; and a substitute first articulation control configured to substitute said first articulation control of said articulation and brake assembly; wherein the substitute first articulation control is longer than the first articulation control; and wherein when the substitute first articulation control knob is attached to the reusable endoscope hand-piece the distance between a centerline of the reusable endoscope hand-piece and a centerline of the substitute first articulation control is greater than the distance between a centerline of the reusable endoscope hand-piece and a centerline of the first articulation control when the first articulation control is attached to the reusable endoscope hand-piece. The first articulation control may support a first articulation control knob. The first articulation control knob may be selectively attachable to either the first articulation control or the substitute first articulation control.

Difference in size of knobs may be a difference in radius, circumference, length, and/or number of lobes.

Lastly, a system is disclosed including a reusable hand-piece and a single-use shaft assembly. The reusable hand-piece selectively attachable to and detachable from the single-use shaft assembly. The reusable hand-piece may include a housing with control switches thereon. An electronic integrated circuit may be positioned within the housing of the reusable hand-piece. The electronic integrated circuit is capable of storing programming of the functions of the control switches and imaging preferences.

Further forms, objects, features, aspects, benefits, advantages, and embodiments of the present disclosure will become apparent from a detailed description and drawings provided herewith.

### BRIEF DESCRIPTION OF THE DRAWINGS

FIG. 1 illustrates a perspective view of an endoscope assembly.

FIG. 2 illustrates an exploded view of the endoscope assembly of FIG. 1.

FIG. 3 illustrates a perspective view of an articulation knob and brake assembly.

FIG. 4 illustrates a partially exploded view of the articulation knob and brake assembly with an up-down articulation control knob, a left-right articulation control knob and a left-right articulation brake knob exploded away from the assembly.

FIG. 5 illustrates a perspective view of an up-down articulation brake outer housing and an up-down articulation shaft.

FIG. 6 illustrates a perspective view of a left-right articulation brake outer housing and a left-right articulation shaft.

FIG. 7 illustrates a perspective view of a reusable hand-piece of the endoscope assembly.

FIG. 8 illustrates an exploded view of the reusable hand-piece of FIG. 7.

FIGS. 9A, 9B and 9C illustrates representative frontal views of the reusable hand-piece with varying housing sizes.

FIGS. 10A, 10B and 10C illustrates a frontal view of the up-down articulation control knob with varying sizes.

FIGS. 11A, 11B and 11C illustrates a frontal view of the up-down articulation control knob with varying numbers of lobes.

FIG. 12 illustrates a side view of the reusable hand-piece demonstrating the distance between the articulation control knob axis of rotation to the backstrap.

FIG. 13 illustrates a rear view of the reusable hand-piece demonstrating the distance between the handle grip centerline and centerline of the articulation control knob lobes.

FIG. 14 illustrates a diagrammatic view of a system, including the reusable hand-piece and articulation knob and brake assembly.

### DETAILED DESCRIPTION OF THE DISCLOSURE

For the purpose of promoting an understanding of the principles of the disclosure, reference will now be made to the embodiments illustrated in the drawings and specific language will be used to describe the same. It will nevertheless be understood that no limitation of the scope of the disclosure is thereby intended. Any alterations and further modifications in the described embodiments, and any further applications of the principles of the disclosure as described herein are contemplated as would normally occur to one skilled in the art to which the disclosure relates. One embodiment of the disclosure is shown in great detail, although it will be apparent to those skilled in the relevant art that some features that are not relevant to the present disclosure may not be shown for the sake of clarity.

With respect to the specification and claims, it should be noted that the singular forms “a”, “an”, “the”, and the like include plural referents unless expressly discussed otherwise. As an illustration, references to “a device” or “the device” include one or more of such devices and equivalents thereof. It also should be noted that directional terms, such as “up”, “down”, “top”, “bottom”, and the like, are used herein solely for the convenience of the reader in order to aid in the reader's understanding of the illustrated embodiments, and it is not the intent that the use of these directional terms in any manner limit the described, illustrated, and/or claimed features to a specific direction and/or orientation.

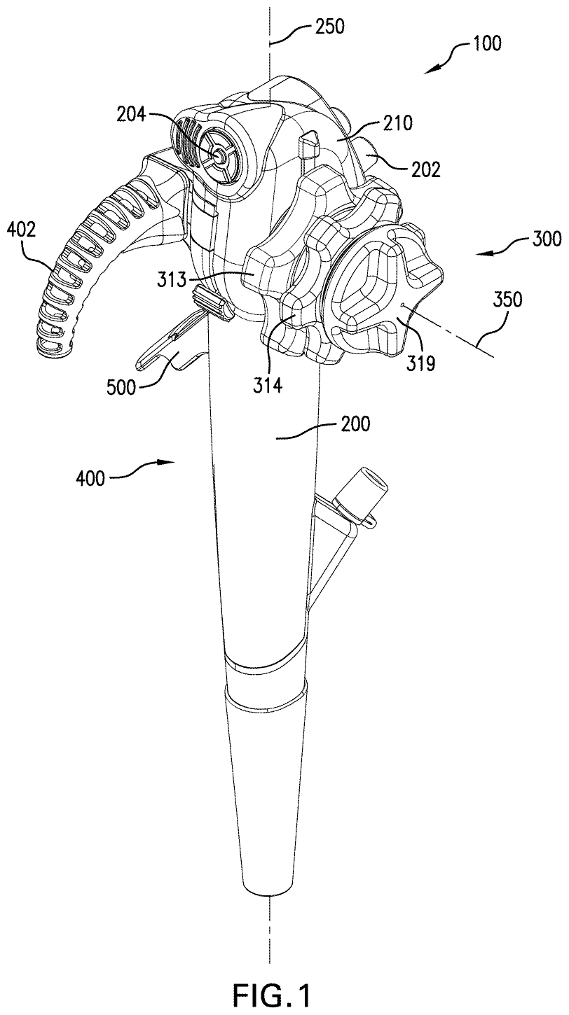

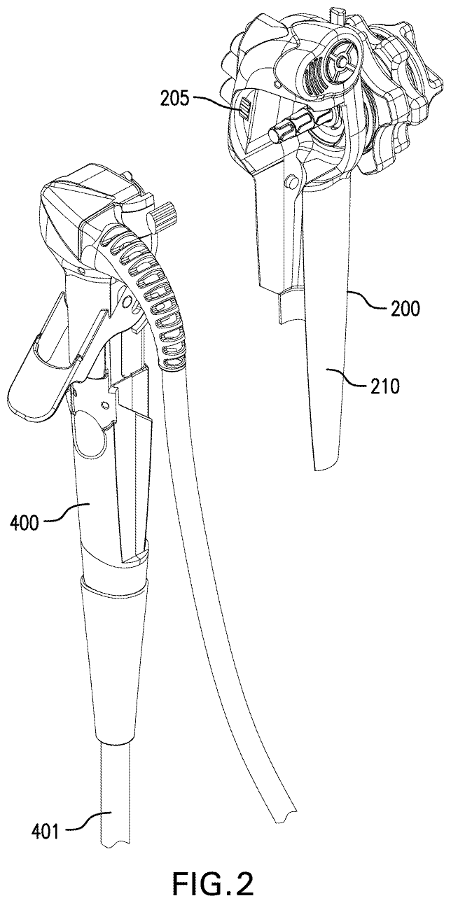

The present disclosure provides an endoscope assembly 100 comprising a modular, customizable reusable hand-piece 200 and a single-use shaft assembly 400. FIG. 1 illustrates the endoscope assembly 100. More particularly, FIG. 1 illustrates the endoscope assembly 100 with the reusable hand-piece 200 attached to the single-use shaft assembly 400. FIG. 2 illustrates the endoscope assembly 100 with the reusable hand-piece 200 detached from the single-use shaft assembly 400. For example, the reusable hand-piece may receive the single-use shaft assembly along a direction transverse (e.g., orthogonal) to a longitudinal axis 250 of the reusable hand-piece. In many instances, when joining the reusable hand-piece and single-use shaft assembly to one another, the single-use shaft assembly will move, relative to the reusable hand-piece, along a direction parallel to an axis of rotation 350 of an articulation knob and brake assembly 300.

The reusable hand-piece 200 is selectively attachable and detachable from the single-use shaft assembly 400 such that the reusable hand-piece may be used serially with a number of single-use shaft assemblies while the single-use shaft assembly can be discarded and/or reconditioned after a single use.

The reusable hand-piece may include a processor and memory that stores settings for controls (e.g., buttons) of the reusable hand-piece and/or for an imaging system connectable to the reusable hand-piece. For example, the reusable hand-piece memory may store user customized settings of the function buttons and image processing (e.g., image capture, image freeze, image color, focus, and/or sensitivity). The processor and/or memory of the reusable hand-piece may be included in an electronic integrated circuit (IC).

The reusable hand-piece and/or single-use shaft assemblies may include a lock for selectively locking the reusable hand-piece and single-use shaft assembly to one another. A latch handle 500 (e.g., a lever) for actuating the latching mechanism connecting the reusable hand-piece to the single-use shaft assembly may be positioned on the reusable hand-piece and/or the single-use shaft assembly. Additional locking mechanisms such as magnets may be used in the alternative or concurrently.

The reusable hand-piece can include a handle portion such as a hand-piece housing 210 supporting an articulation knob and brake assembly 300; control switches 202 for operating light(s), such as light emitting diodes, and/or flush and/or aspiration fluid paths; one or more navigation switches 204 for controlling a console communicating with the endoscope assembly; and/or an electrical connector 205 for electrically coupling the reusable hand-piece to the single-use shaft assembly.

The single-use shaft assembly can include an insertion tube 401 for insertion within the body of a patient, a tubing and electrical connector 402 connected to a console, an access port for selectively accessing a lumen of the single-use shaft assembly (e.g., for taking a sample such as a biopsy) and/or for delivering a fluid through a lumen of the single-use shaft assembly, and/or an electrical connector for electrically coupling the single-use shaft assembly to the reusable hand-piece.

A tubing and/or electrical connector 402, sometimes referred to as an “umbilical”, extends from the endoscope assembly to a cartridge (not shown) that is connectable to a console suitable for use with the endoscope assembly. The tubing and/or electrical connector can extend from the single-use shaft assembly and/or from the reusable hand-piece. In some embodiments, the umbilical may directly connect to the console, eliminating the need for a cartridge.

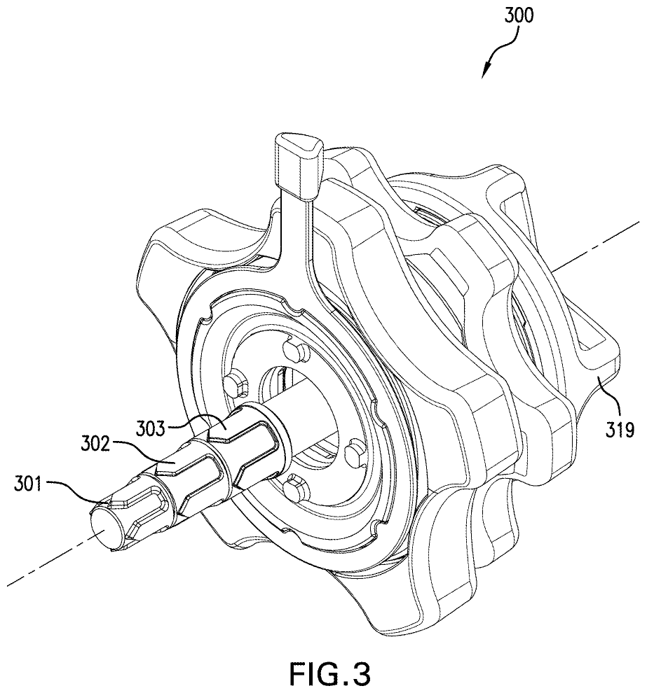

With reference now to FIGS. 3-6, the articulation knob and brake assembly of the reusable hand-piece can include an up-down articulation control knob 313, a left-right articulation control knob 314, an up-down articulation brake and a left-right articulation brake knob 319. During operation, when the reusable hand-piece is attached to the single-use shaft assembly, articulation of the up-down articulation control knob articulates a distal portion of the single-use shaft assembly in a first plane and actuation of the left-right articulation brake knob actuates the distal portion of the single-use shaft assembly in a second plane transverse to the first plane. Preferably, the second plane is orthogonal to the first plane.

When the reusable hand-piece is attached to the single-use shaft assembly, the articulation knob and brake assembly cooperates with the single-use shaft assembly to articulate a distal segment of the single-use shaft assembly and/or resist rotation of one or more articulation knobs relative to the endoscope assembly. For example, one or more articulation shafts 302, 303 and/or brake shafts 301 of the articulation knob and brake assembly may engage a pulley assembly and/or housing of the single-use shaft assembly. One or more shafts of the articulation knob and brake assembly extend through the pulley assembly and engage the housing of the single-use shaft assembly on an opposing side of the pulley assembly from the articulation knob and brake assembly. In some embodiments, shafts 301, 302 and 303 are concentric with a single axis of rotation.

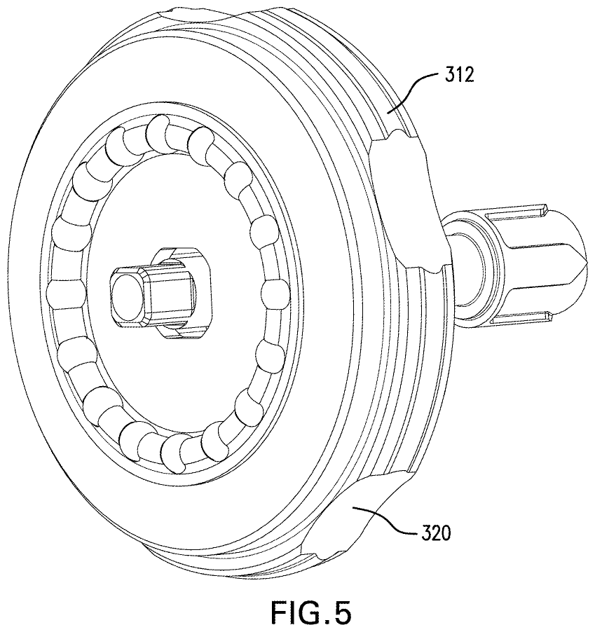

Regarding the up-down articulation control by the articulation knob and brake assembly, the up-down articulation control knob is supported on an up-down articulation brake outer housing 312 (see FIG. 5).

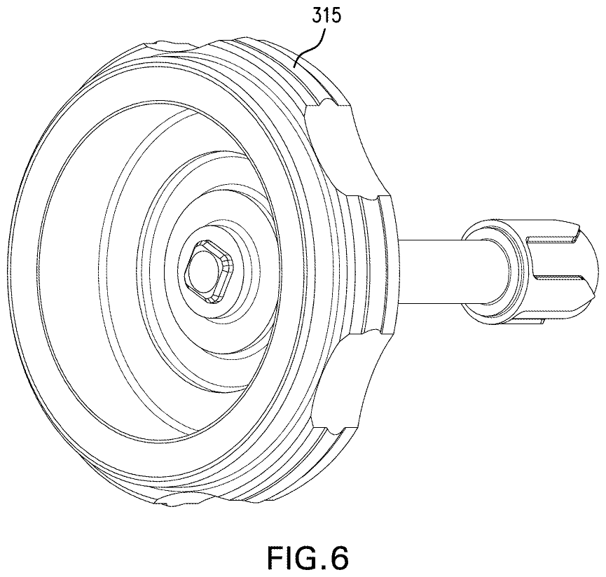

Regarding left-right articulation, the structure and operation of the left-right articulation control knob and brake assembly is the same as the up-down articulation control knob and brake assembly. Similar to the up-down articulation control knob and brake assembly, the left-right articulation control knob is supported by a left-right articulation control knob outer housing 315 (See FIG. 6).

Looking at to FIG. 5, the up-down articulation brake outer housing is shown engaged with and rotationally fixed relative to the up-down articulation shaft. The up-down articulation brake outer housing has a portion 320 arranged to rotationally fix the up-down articulation control knob thereto (e.g., via recesses arranged to receive protrusions of the up-down articulation control knob or vice versa).

With reference to FIG. 6, the left-right articulation brake outer housing and the left-right articulation shaft are shown assembled and rotationally fixed relative to one another.

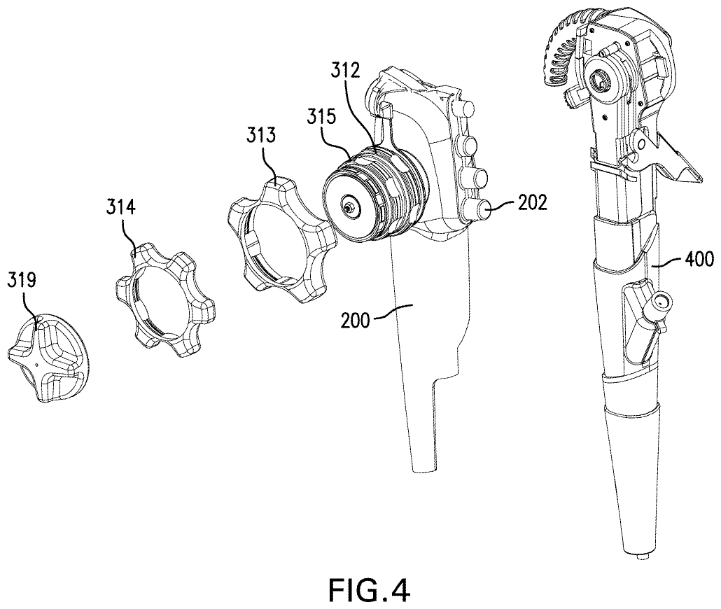

Turning to FIGS. 4, 7 and 8, the up-down articulation control knob 313, the left-right articulation control knob 314 and the left-right articulation brake knob 319 may be detachable (or “snap off”) from the rest of the articulation knob and brake assembly. As discussed above, the up-down articulation brake outer housing has a portion arranged to rotationally fix the up-down articulation control knob thereto (e.g., via recesses arranged to receive protrusions of the up-down articulation control knob or vice versa). Similarly, the left-right articulation brake outer housing is arranged to fix the left-right articulation control knob in the same fashion. The recesses and protrusions arrangement allow the up-down articulation control knob, left-right articulation control knob and left-right articulation brake knob to be easily snapped off or snapped on to the articulation knob and brake assembly. Advantageously, the snap off feature allows the knobs to be removed to facilitate cleaning of the reusable hand-piece between procedures.

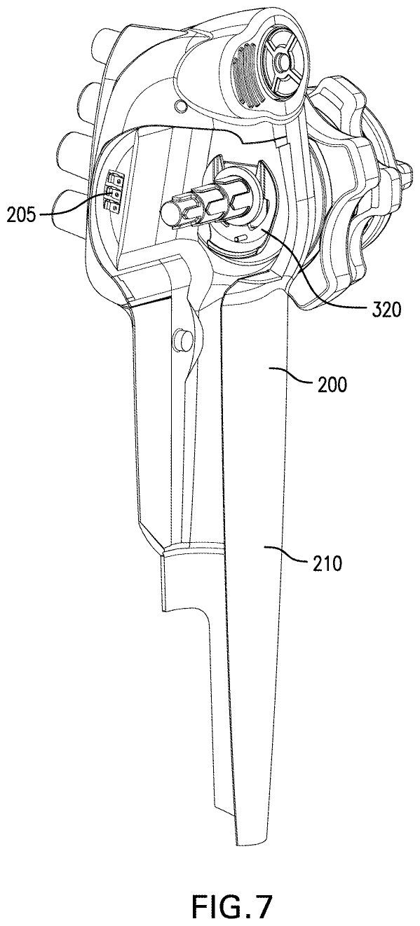

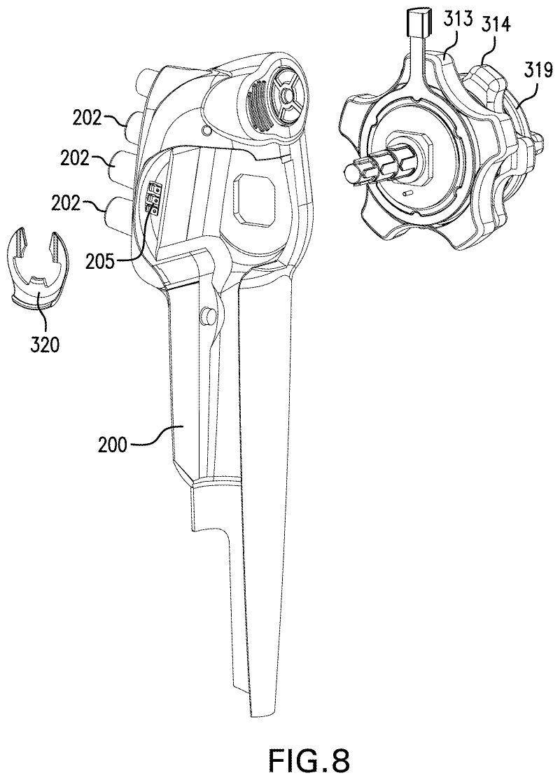

As illustrated in FIGS. 7 and 8, the reusable hand-piece may include a retention clip 320. Retention clip is used to secure articulation knob and brake assembly to the reusable hand-piece. The retention clip may be coupled with the up-down articulation hub to secure the articulation knob and brake assembly. In between procedures, if necessary to facilitate cleaning, the retention clip may be removed from the articulation and brake assembly, separating the articulation and brake assembly from the housing of the reusable hand-piece. Once cleaning is complete, the articulation and brake assembly may be reattached to the housing using the retention clip.

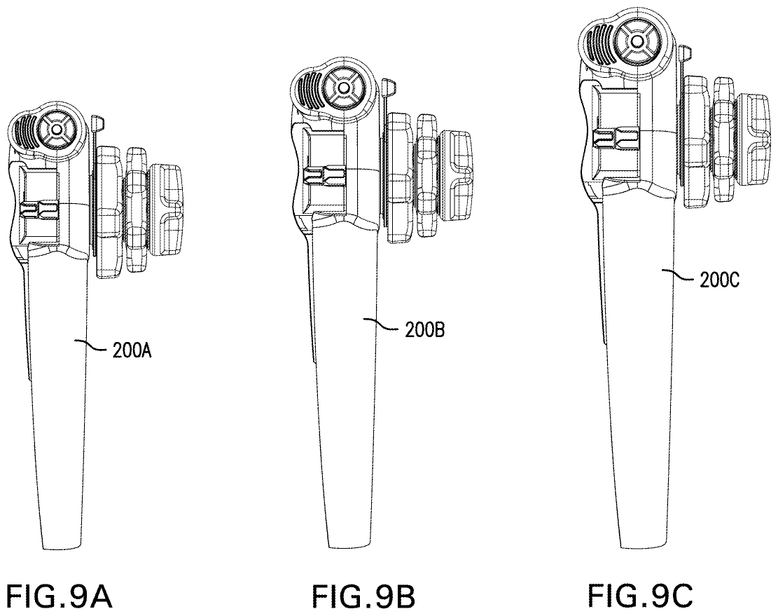

Turning to FIGS. 9A-C, three reusable hand-pieces of different sizes are shown. For illustrative purposes only, FIG. 9A is shown as a “small version” of the reusable hand-piece 200A. FIG. 9B is show as a “medium version” of the reusable hand-piece 200B. In some embodiments, the medium version may be ten percent (10%) larger than the small version of the reusable hand-piece. FIG. 9C illustrates a “large version” of the reusable hand-piece 200C. In some embodiments, the large version may be twenty percent (20%) larger than the small version of the reusable hand-piece. It is understood various other sizes are possible.

Advantageously, the different size reusable hand-piece assemblies allow a user to customize the fit of the endoscope in their hand during a procedure. For example, some users may have smaller hands and may prefer a smaller sized scope in order to perform a procedure more efficiently and effectively.

In some embodiments, both the reusable hand-piece and the single-use shaft assembly may vary in size so that both pieces are of similar size. In other embodiments, only the various reusable hand-piece assemblies may be different sizes. In this embodiment, the different size reusable hand-piece assemblies are configured to attached to or detach from a generic size single-use shaft assembly.

Although illustrated with three different sizes, the reusable hand-piece may be manufactured with only two different sizes, a large and a small version of the reusable hand-piece. In some embodiments, any number of different sizes could be manufactured. The difference in size can be due to different measurables. The difference in size may be a difference in width. The difference in size may be due to a difference in height. The difference in size may be due to a difference in circumference. The difference in size may be a difference in internal volumes.

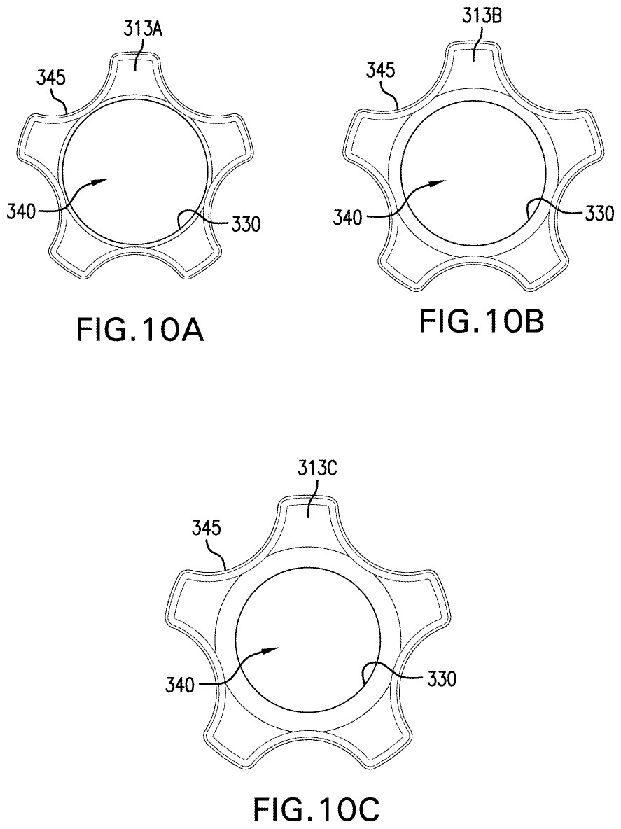

Turning to FIGS. 10A-C, the up-down articulation control knob is illustrated as three different sizes. It is understood that the knob can come in any number of sizes. The up-down articulation control knob is shown for illustrated purposes only. It is understood that all discussion relating to the up-down articulation control knob is equally applicable to the left-right articulation control knob or the left-right articulation brake knob.

For illustrative purposes only, FIG. 10A is shown as a “small version” of the knob 313A. FIG. 10B is show as a “medium version” of the knob 313B. In some embodiments, the medium version may be ten percent (10%) larger than the small version of the knob. FIG. 10C illustrates a “large version” of the knob 313C. In some embodiments, the large version may be twenty percent (20%) larger than the small version of the knob. It is understood various other sizes are possible.

In some embodiments, the knob includes an interior surface 330 defining an opening 340 through the knob. In some embodiments, diameter of the opening or the circumference of the interior surface are the same regardless of the overall size of the knob. The difference in size is due to a difference in the perimeter of an outer surface 345 of the knob.

Advantageously, this allows any size knob to attach to the same up-down articulation brake outer housing or left-right articulation brake outer housing, rather than requiring different sizes of the same up-down articulation brake outer housing or left-right articulation brake outer housing to accommodate the different sized knobs.

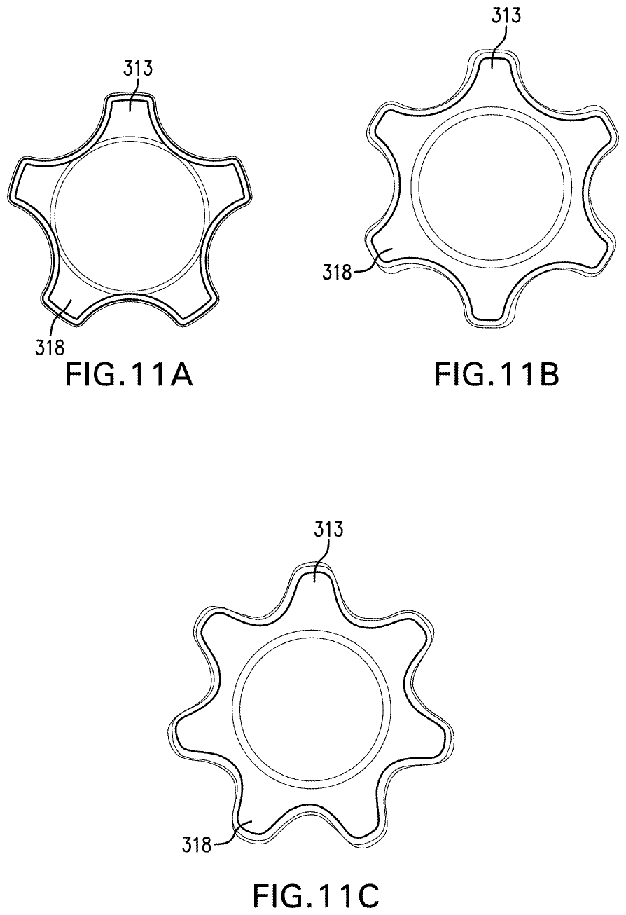

Turning to FIGS. 11A-C, the up-down articulation control knob is illustrated as three different sizes with varying numbers of lobes 318. It is understood that the knob can come with any number of lobes. The up-down articulation control knob is shown for illustrated purposes only. It is understood that all discussion relating to the up-down articulation control knob is equally applicable to the left-right articulation control knob or the left-right articulation brake knob.

For illustrative purposes only, FIGS. 11A-C illustrate the knob with 5, 6 and 7 lobes, respectively. It is understood that any number of lobes is possible. Advantageously, the different number of lobes allows a user to customize the knobs to their preferred number of lobes.

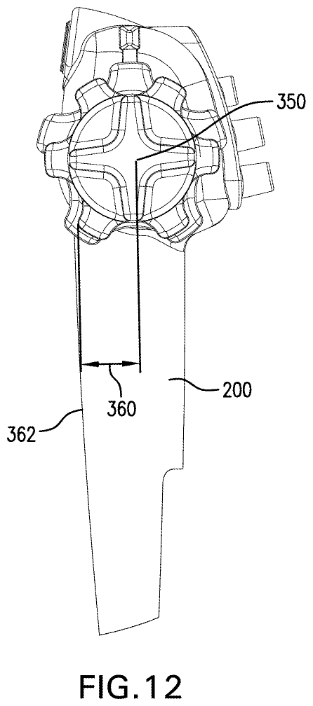

Turning to FIG. 12, a reusable hand piece is illustrated. As shown, there is a distance 360 between the axis of rotation of the articulation control knobs and the backstrap 362. This distance can be varied to fit a user's preference. One way to vary distance is by using different size reusable hand piece assemblies as discussed in relation to FIGS. 9A-C. Smaller reusable hand-piece assemblies will have a smaller distance between the axis of rotation of the articulation control knobs and the backstrap.

In other embodiments, distance can be altered by using up-down articulation brake outer housings or left-right articulation brake outer housings of different sizes. Additionally, the housings can be manufactured to laterally offset the knobs to either increase or decrease the distance between the axis of rotation of the articulation control knobs and the backstrap.

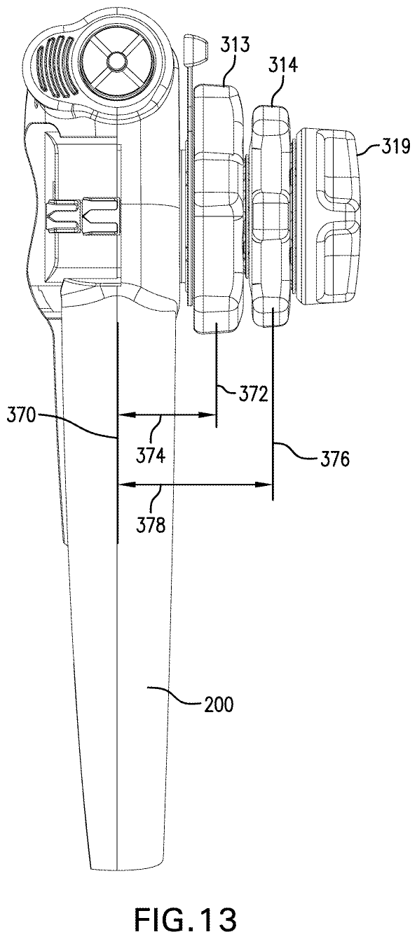

Turning to FIG. 13, a reusable hand piece is illustrated. A centerline 370 of the reusable hand-piece and a centerline 372 of the up-down articulation control knob of the first reusable hand-piece is distance 374 apart. A centerline of the reusable hand-piece and a centerline 376 of the left-right articulation control knob of the first reusable hand-piece is distance 378 apart. These distances can be varied to fit a user's preference. One way to vary distance is by using different size reusable hand piece assemblies as discussed in relation to FIGS. 9A-C. Smaller reusable hand-piece assemblies will have a smaller distance between the axis of rotation of the articulation control knobs and the backstrap. In some embodiments, distance can be altered by using up-down articulation brake outer housings or left-right articulation brake outer housings of different sizes.

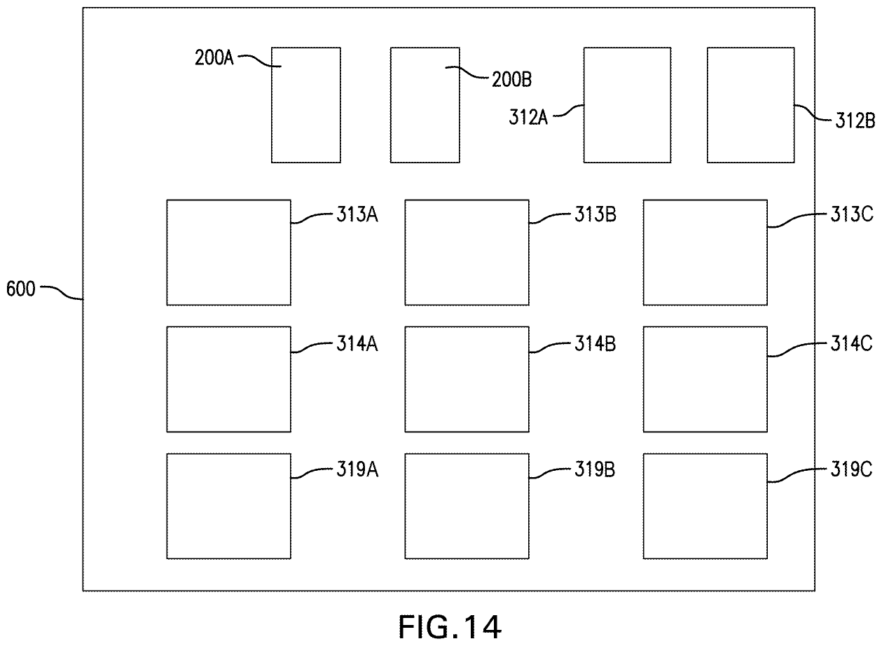

FIG. 14 illustrates a system, such as a product line. The system may include one or more components packaged together or separately in sterilely sealed medical packaging. The system may include a first reusable hand-piece 200A, a second reusable hand-piece 200B. In some embodiments, the first reusable hand-piece is a different size than the second reusable hand-piece. As previously discussed, both the first reusable hand-piece and the second reusable hand-piece may be selectively attachable and detachable to a single-use shaft assembly.

As illustrated the system may further include a first up-down articulation control knob 313A, a second up-down articulation control knob 313B and/or a third up-down control knob 313C. The up-down articulation control knobs are preferably different sizes and/or have different numbers of lobes. Additionally, the system may include a first left-right articulation control knob 314A, a second left-right articulation control knob 314B and/or a third left-right control knob 314C. The left-right articulation control knobs are preferably different sizes and/or have different numbers of lobes. Finally, the system may also include a first left-right articulation brake knob 319A, a second left-right articulation brake knob 319B and/or a third left-right articulation brake knob 319C. The left-right articulation brake knobs are preferably different sizes.

The system may include up-down articulation brake outer housings 312A and 312B or left-right articulation brake outer housings (not illustrated) of different sizes.

The system may include any combination of reusable hand-piece assemblies, up-down articulation control knobs, left-right articulation control knobs, left-right articulation brake knobs, up-down articulation brake outer housings and/or left-right articulation brake outer housings.

While the disclosure has been illustrated and described in detail in the drawings and foregoing description, the same is to be considered as illustrative and not restrictive in character, it being understood that only the preferred embodiment has been shown and described and that all changes, equivalents, and modifications that come within the spirit of the disclosures defined by following claims are desired to be protected. All publications, patents, and patent applications cited in this specification are herein incorporated by reference as if each individual publication, patent, or patent application were specifically and individually indicated to be incorporated by reference and set forth in its entirety herein.

### Clauses

The following numbered clauses set out specific embodiments that may be useful in understanding the present disclosure:

1. A system, comprising: a first reusable endoscope hand-piece; a second reusable endoscope hand-piece, wherein the first reusable endoscope hand-piece is a different size and/or has at least one different feature than the second reusable endoscope hand-piece; and wherein both the first reusable endoscope hand-piece and the second reusable endoscope hand-piece are alternatively and selectively attachable and detachable to a single-use endoscope shaft assembly and configured to articulate a patient-insertable portion of the single-use endoscope shaft assembly when it is inserted within a body of a patient.

2. The system of clause 1, wherein the first reusable endoscope hand-piece and the second reusable endoscope hand-piece each include a housing and an articulation assembly, and wherein the articulation assembly includes a first articulation control for articulating the patient-insertable portion of the single-use endoscope shaft assembly along a first direction.

3. The system of clause 2, wherein a distance between a backstrap and an axis of rotation of the first articulation control of the first reusable endoscope hand-piece is greater than a distance between a backstrap and an axis of rotation of the first articulation control of the second reusable hand-piece.

4. The system of any one of clauses 2-3, wherein a distance between a centerline of the first articulation control and a centerline of the handle portion of the first reusable endoscope hand-piece is greater than a distance between the centerline of the first articulation control and a centerline of the handle portion of the second reusable hand-piece.

5. The system of any one of clauses 2-4, further comprising a second articulation control for articulating the patient-insertable portion of the single-use endoscope shaft assembly along a second direction that is transverse to the first direction

6. The system of clause 5, wherein the first articulation control is a first articulation control knob and the second articulation control is a second articulation control knob; and wherein the first articulation control knob and the second articulation control knob are selectively attachable and detachable to the first reusable endoscope hand-piece.

7. The system of clause 6, further comprising: a substitute first articulation control knob alternatively and selectively attachable and detachable to the first reusable endoscope hand-piece instead of said first articulation control knob; and wherein the substitute first articulation control knob has more outwardly-extending lobes than said first articulation control knob

8. The system of clause 7, further comprising: a substitute second articulation control knob alternatively and selectively attachable and detachable to the first reusable endoscope hand-piece instead of said second articulation control knob; and wherein the substitute second articulation control knob has more outwardly-extending lobes than said second articulation control knob

9. The system of any preceding clause, wherein the first reusable endoscope hand-piece is a different size than the second reusable endoscope hand-piece; and wherein the difference in size is a difference in cross-sectional area of a portion arranged to contact a user's palm during operation of the reusable endoscope hand-piece.

10. The system of any preceding clause, wherein the first reusable endoscope hand-piece is a different size than the second reusable endoscope hand-piece; and wherein the difference in size is a difference in height.

11. The system of any preceding clause, wherein the first reusable endoscope hand-piece is a different size than the second reusable endoscope hand-piece; and wherein the difference in size is a difference in total volume.

12. The system of any preceding clause, wherein the first reusable endoscope hand-piece and the second reusable endoscope hand-piece each include an electronic integrated circuit storing settings for control and/or imaging functions of an assembled reusable endoscope hand-piece and single-use endoscope shaft assembly.

13. The system of clause 12, wherein settings stored in the electronic integrated circuit of the first reusable endoscope hand-piece are different from those in the second reusable endoscope hand-piece.

14. The system of any preceding clause, wherein both the first reusable endoscope hand-piece and the second reusable endoscope hand-piece are alternatively and selectively attachable and detachable to a second single-use endoscope shaft assembly; and wherein the single-use endoscope shaft assembly is different in size and/or has at least one different feature than the second single-use endoscope shaft assembly.

15. The system of clause 14, wherein the second single-use endoscope shaft assembly is longer than the single-use endoscope shaft assembly.

16. The system of any one of clauses 14-15, wherein the second single-use endoscope shaft assembly has a greater insertion tube diameter than the single-use endoscope shaft assembly.

17. The system of any preceding clause, wherein the system is a line of products.

18. A system, comprising: a reusable endoscope hand-piece and a single-use endoscope shaft assembly; the reusable endoscope hand-piece selectively attachable to and detachable from the single-use endoscope shaft assembly; the reusable endoscope hand-piece including a housing and an articulation and brake assembly, wherein the articulation and brake assembly includes a first articulation control, a second articulation control, and an articulation brake for selectively locking rotation of said second articulation control relative to the housing of said reusable endoscope hand-piece; and a substitute first articulation control configured to substitute said first articulation control of said articulation and brake assembly; wherein the substitute first articulation control is longer than the first articulation control; and wherein when the substitute first articulation control is attached to the reusable endoscope hand-piece a distance between a centerline of the reusable endoscope hand-piece and a centerline of the substitute first articulation control is greater than a distance between a centerline of the reusable endoscope hand-piece and a centerline of the first articulation control when the first articulation control is attached to the reusable endoscope hand-piece.

19. The system of clause 18, wherein the first articulation control supports a first articulation control knob.

20. The system of clause 19, wherein the first articulation control knob is selectively attachable to either the first articulation control or the substitute first articulation control.

1. A system, comprising:
a first reusable endoscope hand-piece;
a second reusable endoscope hand-piece, wherein the first reusable endoscope hand-piece is a different size and/or has at least one different feature than the second reusable endoscope hand-piece; and
wherein both the first reusable endoscope hand-piece and the second reusable endoscope hand-piece are alternatively and selectively attachable and detachable to a single-use endoscope shaft assembly and configured to articulate a patient-insertable portion of the single-use endoscope shaft assembly when it is inserted within a body of a patient.

2. The system of claim 1, wherein the first reusable endoscope hand-piece and the second reusable endoscope hand-piece each include a housing and an articulation assembly, and wherein the articulation assembly includes a first articulation control for articulating the patient-insertable portion of the single-use endoscope shaft assembly along a first direction.

3. The system of claim 2, wherein a distance between a backstrap and an axis of rotation of the first articulation control of the first reusable endoscope hand-piece is greater than a distance between a backstrap and an axis of rotation of the first articulation control of the second reusable hand-piece.

4. The system of claim 2, wherein a distance between a centerline of the first articulation control and a centerline of the handle portion of the first reusable endoscope hand-piece is greater than a distance between the centerline of the first articulation control and a centerline of the handle portion of the second reusable hand-piece.

5. The system of claim 2, further comprising a second articulation control for articulating the patient-insertable portion of the single-use endoscope shaft assembly along a second direction that is transverse to the first direction.

6. The system of claim 5, wherein the first articulation control is a first articulation control knob and the second articulation control is a second articulation control knob; and
wherein the first articulation control knob and the second articulation control knob are selectively attachable and detachable to the first reusable endoscope hand-piece.

7. The system of claim 6, further comprising:
a substitute first articulation control knob alternatively and selectively attachable and detachable to the first reusable endoscope hand-piece instead of said first articulation control knob; and
wherein the substitute first articulation control knob has more outwardly-extending lobes than said first articulation control knob.

8. The system of claim 7, further comprising:
a substitute second articulation control knob alternatively and selectively attachable and detachable to the first reusable endoscope hand-piece instead of said second articulation control knob; and
wherein the substitute second articulation control knob has more outwardly-extending lobes than said second articulation control knob.

9. The system of claim 1, wherein the first reusable endoscope hand-piece is a different size than the second reusable endoscope hand-piece; and
wherein the difference in size is a difference in cross-sectional area of a portion arranged to contact a user's palm during operation of the reusable endoscope hand-piece.

10. The system of claim 1, wherein the first reusable endoscope hand-piece is a different size than the second reusable endoscope hand-piece; and
wherein the difference in size is a difference in height.

11. The system of claim 1, wherein the first reusable endoscope hand-piece is a different size than the second reusable endoscope hand-piece; and
wherein the difference in size is a difference in total volume.

12. The system of claim 1, wherein the first reusable endoscope hand-piece and the second reusable endoscope hand-piece each include an electronic integrated circuit storing settings for control and/or imaging functions of an assembled reusable endoscope hand-piece and single-use endoscope shaft assembly.

13. The system of claim 12, wherein settings stored in the electronic integrated circuit of the first reusable endoscope hand-piece are different from those in the second reusable endoscope hand-piece.

14. The system of claim 1, wherein both the first reusable endoscope hand-piece and the second reusable endoscope hand-piece are alternatively and selectively attachable and detachable to a second single-use endoscope shaft assembly; and
wherein the single-use endoscope shaft assembly is different in size and/or has at least one different feature than the second single-use endoscope shaft assembly.

15. The system of claim 14, wherein the second single-use endoscope shaft assembly is longer than the single-use endoscope shaft assembly.

16. The system of claim 14, wherein the second single-use endoscope shaft assembly has a greater insertion tube diameter than the single-use endoscope shaft assembly.

17. The system of claim 1, wherein the system is a line of products.

18. A system, comprising:
a reusable endoscope hand-piece and a single-use endoscope shaft assembly;
the reusable endoscope hand-piece selectively attachable to and detachable from the single-use endoscope shaft assembly;
the reusable endoscope hand-piece including a housing and an articulation and brake assembly, wherein the articulation and brake assembly includes a first articulation control, a second articulation control, and an articulation brake for selectively locking rotation of said second articulation control relative to the housing of said reusable endoscope hand-piece; and
a substitute first articulation control configured to substitute said first articulation control of said articulation and brake assembly;
wherein the substitute first articulation control is longer than the first articulation control; and
wherein when the substitute first articulation control is attached to the reusable endoscope hand-piece a distance between a centerline of the reusable endoscope hand-piece and a centerline of the substitute first articulation control is greater than a distance between a centerline of the reusable endoscope hand-piece and a centerline of the first articulation control when the first articulation control is attached to the reusable endoscope hand-piece.

19. The system of claim 18, wherein the first articulation control supports a first articulation control knob.

20. The system of claim 19, wherein the first articulation control knob is selectively attachable to either the first articulation control or the substitute first articulation control.
